(19) United States
(12) Patent Application Publication
(10) Pub. No.: US 2008/0108947 A1
(43) Pub. Date: May 8, 2008

(54) CATHETER HOLDER APPARATUS AND METHOD

(76) Inventor: Bruce Crawford, Reno, NV (US)

Correspondence Address: HOLLAND & HART, LLP, P.O BOX 8749, DENVER, CO, 80201, US

(21) Appl. No.: 11/868,384

(22) Filed: Oct. 5, 2007

Related U.S. Application Data
(60) Provisional application No. 60/849,695, filed on Oct. 5, 2006.

(51) Int. Cl. A61M 25/02

(52) U.S. Cl. 604/174

(57) ABSTRACT

There is disclosed a catheter holding device for use with a urethral catheter in a urodynamic testing procedure. In one embodiment, the device includes a catheter holding arm for holding the urethral catheter directly in front of the urethra during the urodynamic testing procedure. In another embodiment, there is disclosed a method of holding a urethral catheter. The method includes attaching a base portion of a urodynamic catheter holder to an inner thigh of a patient; extending a catheter holding arm away from the base portion, toward an external urethral meatus of the patient; and securing a catheter to the catheter holding arm at a position substantially in alignment with a urinary lumen of the patient. Other embodiments are also disclosed.

### PRIORITY CLAIM

This patent application claims the benefit of pending prior U.S. Provisional Patent Application Ser. No. 60/849,695, filed Oct. 5, 2006, by Bruce S. Crawford for NOVEL CATHETER HOLDER, which patent application is hereby incorporated herein.

### FIELD OF THE INVENTION

The present invention relates to the field of catheter securement and stabilizing devices. More particularly, and in various embodiments, the device has applications in the field of urodynamic testing.

### BACKGROUND

Many instruments have been described to hold catheters in a secure position within the body. Many such devices have been used to secure urethral urinary catheters to the patient's body. One example of such a device is the Dale catheter holder (Dale Medical Products Inc., Plainville, Mass.). This device includes an elastic leg strap and a catheter securement mechanism utilizing a “duel patented locking device” that holds the catheter in place. U.S. Pat. No. 4,569,348, issued to Hasslinger, and assigned to Velcro USA of Manchester, N.H., discloses a leg strap catheter holder using hook-and-loop fasteners and adhesive materials to secure a conventional catheter to a patient's leg.

One specific use of urinary catheters is for urodynamic testing. Typically, this testing is an office or hospital based procedure in which bladder storage and emptying is studied. Urodynamic testing is a term that describes a number of individual tests such as uroflometry, cystometrogram, urethral pressure profilemetry, bladder pressure voiding studies, abdominal pressure voiding studies. Often several, or all of these procedures, are performed in one setting to provide a comprehensive assessment of the patients bladder function. In general, these tests involve the measurement of pressure at various sites such as the bladder, urethra, rectum or vagina using specialized catheters. These catheters are used to conduct a signal to a measurement device that reports and records the pressure within the catheterized organ. The signal may be water pressure, an induced electrical current, a fiber optic signal, or air pressure. Often fluoroscopy is used during the procedure to visualize the bladder and urethra.

In the course of performing a cystometrogram, a urethral catheter is placed through the patient's urethra into the urinary bladder. Next, the cystometrogram typically includes a retrograde fill of the bladder with sterile fluid. The pressure in the bladder is measured as filling takes place and the patient is asked to described various sensations of fullness as the test proceeds. Often during the cystometrogram, the patient is asked to perform various prevocational maneuvers, such as coughing and valsalva, to test for stress urinary incontinence. While performing this study, the catheter is generally secured to the patients thigh or abdomen using adhesive tape.

In the course of performing a urethral pressure profile, the urethral catheter is drawn through the urethra as a real time pressure recording is made. This procedure requires removal of the catheter from the patient's thigh or abdomen so it can be moved telescopically through the urethra as the pressure measurements are made.

In the course of performing abdominal pressure voiding studies and bladder pressure voiding studies, i.e., micturition studies, the catheters are again secured to the patients thigh or abdomen. The patient is required to void with the catheters in place so as to record the pressure changes within the bladder and abdominal cavity, e.g., via a vaginal catheter or a rectal catheter, as the patient voids.

The current practice of taping the catheter to the patient's leg or abdomen has several disadvantages, which may include, but are not limited to, (1) bowing of the catheter promotes expulsion of the catheter from the urethra; (2) the need to un-tape and re-tape the catheter during the procedure; (3) irritation and discomfort due to the use of a relatively large amount of tape and removal of the tape; and (4) difficulty to remove adhesive left by the tape from the surface of reusable catheters, e.g., microtip catheters. Instead of taping a catheter, another securing device may be desired for urodynamic testing procedures and other catheter securement applications.

### SUMMARY

In an embodiment, a catheter holding device, for use with a urethral catheter in a urodynamic testing procedure, comprises a catheter holding arm for holding the urethral catheter directly in front of the urethra during the urodynamic testing procedure.

In another embodiment, a method of holding a urethral catheter comprises attaching a base portion of a urodynamic catheter holder to an inner thigh of a patient; extending a catheter holding arm away from the base portion, toward an external urethral meatus of the patient; and securing a catheter to the catheter holding arm at a position substantially in alignment with a urinary lumen of the patient.

In another embodiment, a catheter holding device for use with a urethral catheter in a urodynamic testing procedure comprises a base portion having a first side and a second side, the first side configured for attachment to an inner thigh of a patient; and a catheter holding arm having a first end and a second end, the first end extending from the second side of the base portion, the second end selectively positionable with respect to the base portion, and a closure portion for attaching the urethral catheter to the catheter holding arm, the closure disposed away from the first end, whereby the catheter holding arm attaches to the urethral catheter in front of the urethra during the urodynamic testing procedure.

There are other embodiments and aspects of them. They will become apparent as this specification proceeds. In this regard, it is to be understood that an embodiment of the present invention need not address issues noted in the background or include features set forth in this brief Summary of certain aspects of this specification.

### BRIEF DESCRIPTION OF THE DRAWINGS

The preferred and other embodiments are shown in the accompanying Figures in which:

FIG. 1 illustrates a female perineum;

FIGS. 2A and 2B illustrate a urodynamic catheter holder;

FIG. 2C illustrates an exploded view of a catheter holding arm of the urodynamic catheter holder;

FIG. 3 illustrates catheters held in place by the urodynamic catheter holder of FIGS. 2A and 2B, positioned on a female patient's inner thigh;

FIG. 4A illustrates a securing member with an adhesive backing used to secure catheter to a base portion of the urodynamic catheter holder;

FIG. 4B illustrates the securing member of FIG. 4A with hooks in engagement with the base portion of the holder, and the adhesive backing in attachment with the catheter;

FIG. 5 illustrates catheters held in place by the urodynamic catheter holder of FIGS. 2A and 2B, positioned on a male patient's inner thigh; and

FIG. 6 illustrates an exemplary method of holding a urethral catheter.

### DETAILED DESCRIPTION

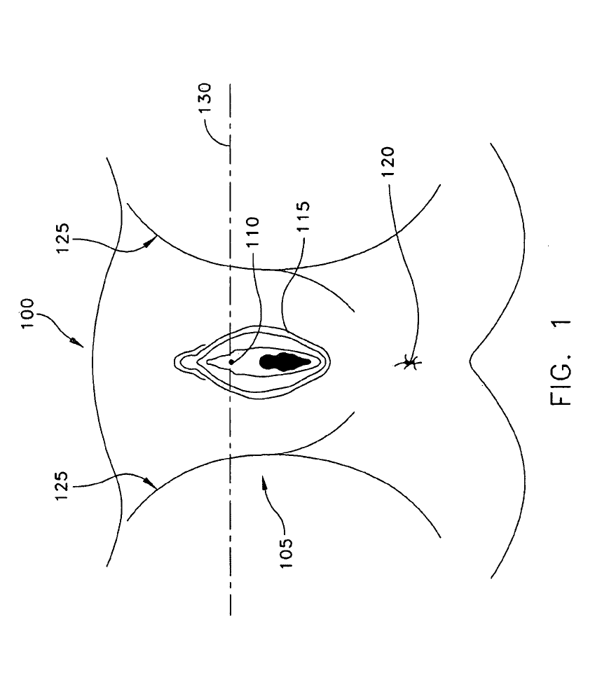

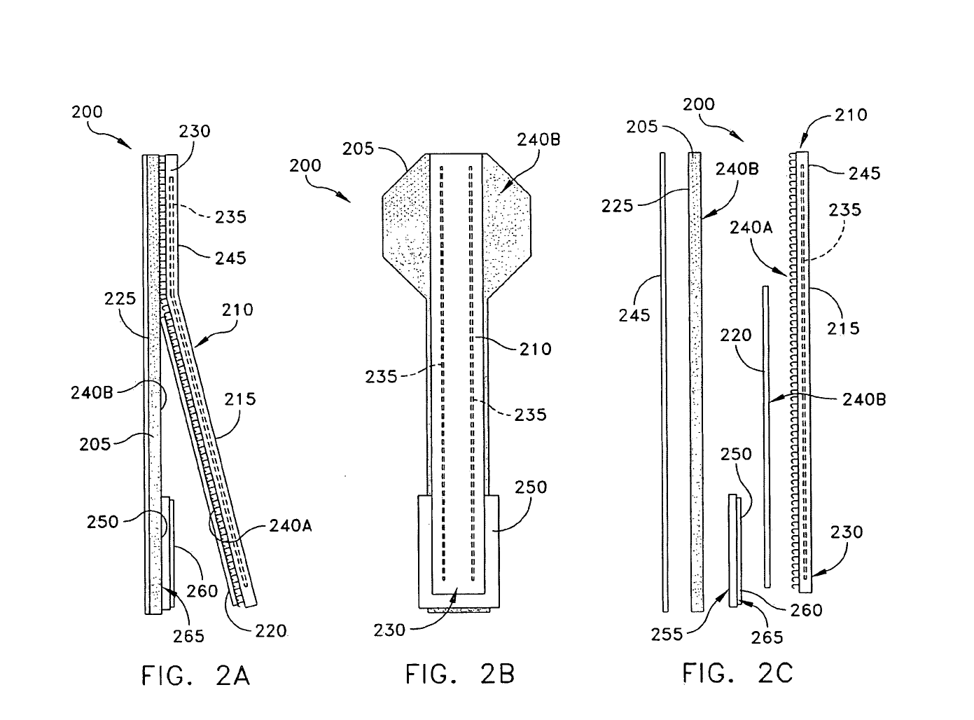

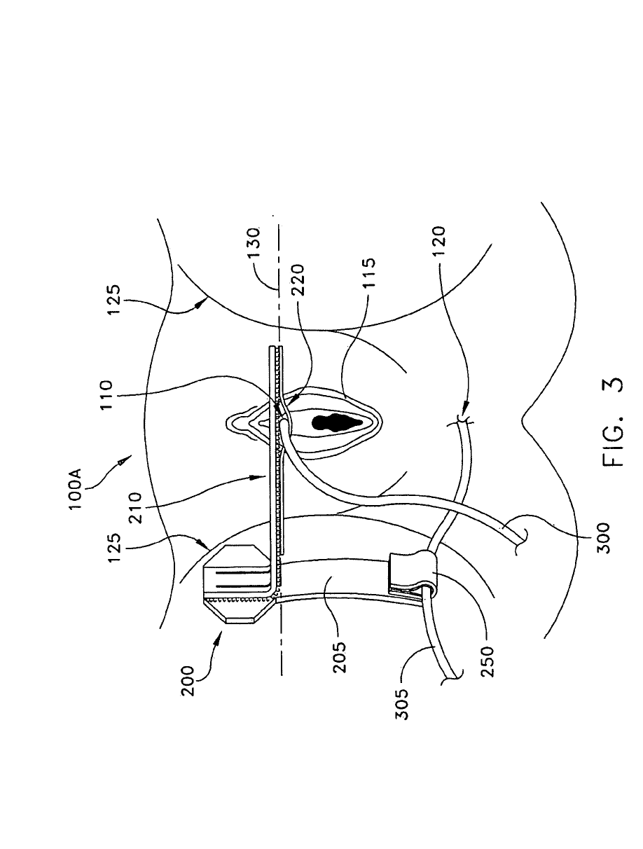

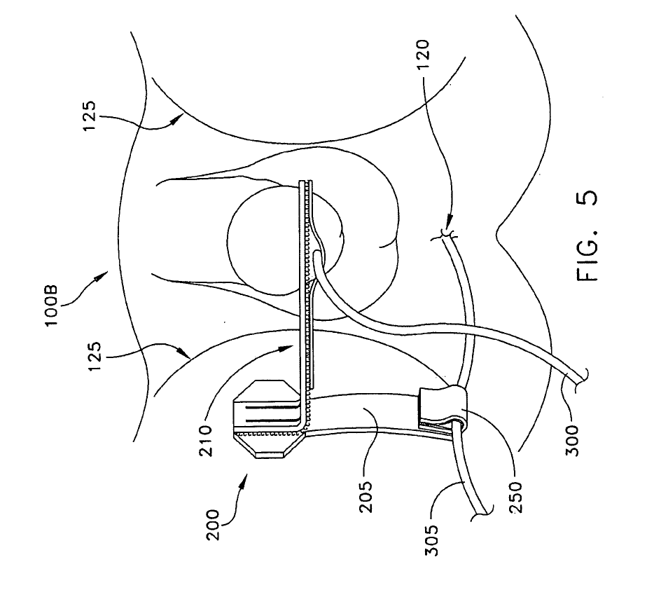

FIG. 1 illustrates a female perineum 100A of a patient including two thighs 105, an external urethral meatus 110, a labia majus 115, an anus 120, a thigh crease 125, and a midline 130. Referring to FIGS. 2A-2C and in an embodiment, a urodynamic catheter holder 200, which is also referred to as an “UCH” 200, is a device designed to hold one or more catheters 300, 305 (FIGS. 3 and 5). UCH 200 may be used in the context of urodynamic testing of a patient. In various embodiments, UCH 200 may be disposable or reusable. UCH 200 may include a base portion 205 designed to be secured to a patient's inner thigh 105, and a catheter holding arm 210 designed to extend medially across a midline 130 immediately in front of external urethral meatus 110.

In an embodiment, catheter holding arm 210 includes a malleable material 230 and may be configured from a position in parallel with base portion 205 to a position at about a 90° angle to base portion 205. In one embodiment, malleable material 230 may include 16-18 gauge wires 235. A semi-rigid portion 215 and a closure portion 220 may be provided by catheter holding arm 210. Semi-rigid portion 215 may be selectively positionable and selectively attachable to base portion 205. Closure portion 220 may be selectively attachable to semi-rigid portion 215 so as to allow selective positioning of a catheter with respect to urethral meatus. Catheter holding arm 210 receives and securely holds urethral catheter 300 as it extends from external urethral meatus 110. By securing catheter 300 in line with the patient's urethral lumen, for example, bowing of catheter 300 can be eliminated if desired.

In an embodiment, hook-and-loop material 240A, 240B is disposed on semi-rigid portion 215 of catheter holding arm 210, and on base portion 205 and closure portion 220.

Catheter holding arm 210 can readily release and re-secure urethral catheter 300, so that it may be easily repositioned during the course of a urodynamic procedure. Catheter holding arm 210 can hold urethral catheter 300 with sufficient force to prevent movement of catheter 300 relative to patient 100 during the various prevocational maneuvers used during urodynamic testing, e.g., valsalva and coughing. Catheter holding arm 210 typically allows catheter 300 to advance further into, or be withdrawn from, the bladder by pulling or pushing catheter 300 manually with a reasonable amount of force.

Referring now to FIGS. 2A-2C, 3 and 5, in one particular embodiment, a second catheter 305 (FIGS. 3 and 5) may be secured to the base portion 205 using a securing portion 250, such as a ½″×1″-2″ segment of adhesive backed, and hook exposed hook-and-loop material.

Base portion 205 of UCH 200 may be secured to the patient's inner thigh 105 in numerous ways, some of which are discussed herein below. Base element 205 may be positioned on the patient's thigh to allow malleable arm 210 to be bent in a medial direction, such that it extends medially across midline 130 at the level of external urethral meatus 220. In one embodiment, base element 205 may be secured to the patient's inner thigh 105 with an adhesive strip 225. In one embodiment, base portion 205 may be positioned just lateral to the patient's right left thigh crease 125. Base portion 205 may be designed to provide a point of securement for a second urodynamic catheter 305 or other type of catheter 305. In one embodiment, second catheter 305 may include a vaginal or rectal catheter used to measure abdominal pressure.

UCH 200 can be readily removed from the patient's inner thigh 105 at the conclusion of the procedure. In some embodiments, UCH 200 is secured to the patient's thigh 105 with a single ½″×2-5″ adhesive strip 225 that is readily removed with or without the use of an adhesive remover swab.

UCH 200 may be produced as a disposable or reusable medical instrument. In an embodiment, UCH 200 is fabricated from hook-and-loop material with a medical grade adhesive backing 225 as an inexpensive disposable single use only device. In one embodiment, the malleable arm 210 may contain two segments of 16-18 gauge wire 235, or other suitable malleable material 235, along the length of its long axis. Malleable material 230 may be sandwiched between the back surface of a segment of hook-and-loop material 240A, 240B and a fabric adhesive backing 245. In various embodiments of UCH 200, hook-surface 240A or loop-surface 240B may be interchanged while maintaining a mating relationship with a compatible hook-surface 240A or loop-surface 240B at necessary points of adherence for assembly.

Referring now to FIG. 2A, a urodynamic catheter holder 200, which may be sterilized, is packaged and delivered for use in packaged form. After unpacking, the urodynamic catheter holder 200 may be applied to, for example, a patient's thigh 105 by removing adhesive backing from adhesive strip 225 of base portion 205. Loop surface 240B may be provided on base portion 205. Malleable urethral catheter arm 210 may be disposed parallel to adhesive strip 225 of base element as UCH device 200 is applied to the patient's thigh 105. FIG. 2C illustrates closure portion 220 having an adhesive backing over loop surface 240B that covers the wire elements 240A of the malleable urethral catheter arm 210. In one embodiment, the urethral catheter arm is affixed to base portion 205 using hook and loop technology. Optionally, a variety of methods could be used to attach the various elements of the UCH to each other including sewing, gluing, and melting. It should be appreciated that, with the use of hook-and-loop closures and adhesive materials, the UCH 200 can be completely assembled quickly and easily without the need for sewing the various parts together. In FIG. 2A, a segment of hook-and-loop material 250 is matingly compatible with the hook-and-loop surface of catheter holding arm 210 so that the two layers can be pressed together around the urethral catheter.

FIG. 3 illustrates UCH 200 affixed to a patient's inner thigh with the malleable urethral catheter arm 210 bent into a position typically used in the course of urodynamic testing. The urethral catheter arm 210 contains malleable material 230 (FIG. 2), which may be bent into a favorable position before, or during, the procedure. Because the malleable material 230 is sufficiently stiff, urethral catheter arm 210 will be held in this favorable position as the procedure is performed. The malleable arm 210 may include an approximately 3″ segment of hook-and-loop closure material 240A, and is shown with a surface with hooks exposed. Urethral catheter 300 may pass between semi-rigid portion 215 and closure portion 220 of catheter holding arm 210 as it emerges from urethra 110 (FIG. 1).

In one embodiment, securing portion 250 may include an approximately 2″ piece of hook-and-loop material with adhesive backing 260. Securing portion 250 may be used to secure a second catheter 305 to base strip 205, of the UDC 200. Adhesive backing of securing porting 250 may be used to wrap around the second catheter 305 such that securing portion 250 is effectively folded in half around catheter. As such, hook surface 255 of securing portion 250 may be positioned away from catheter 305 and may be used to secure the assembly to loop surface 240B of base strip 205. Alternatively, second catheter 305 may be sandwiched loop-surface 240B of base strip 205 and hook-surface of securing portion 250. This configuration can reduce or even eliminate need for adhesive backing 260 on securing portion 250.

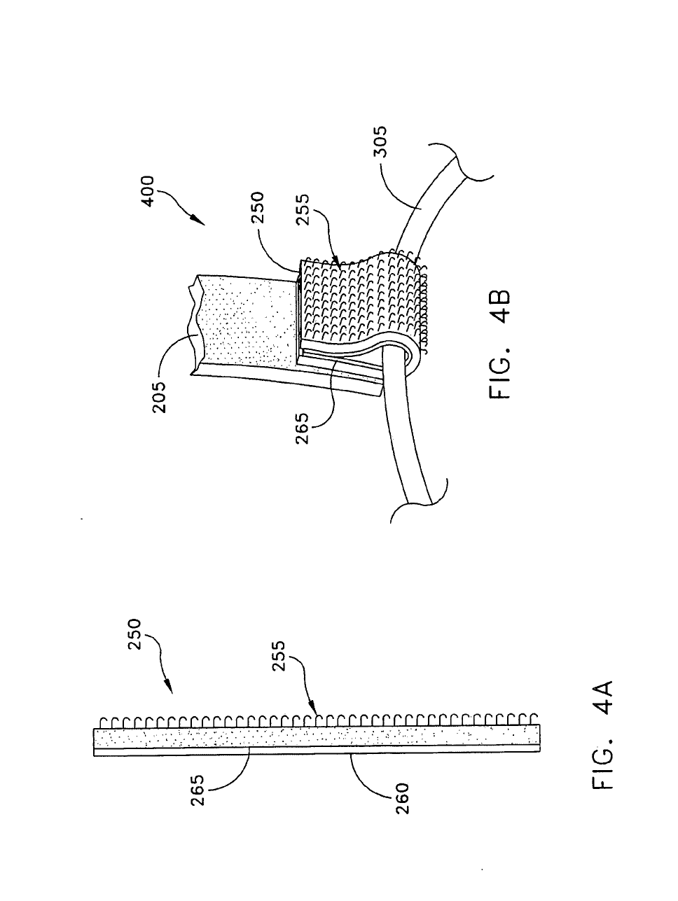

FIGS. 4A and 3B depict second catheter holder 250 as used in an embodiment to secure second catheter 305 to base portion 205. Adhesive strip backing 260, when peeled away from adhesive coating 265, allows the operator to fold adhesive surface 265 around catheter 305, and, thereafter, attach the exposed surface of hook-and-loop material 255 to a matingly compatible surface of hook-and-loop material 240 of base portion 206

Referring to FIG. 5, a male perineum 100B of a patient includes two thighs 105, external urethral meatus 110, anus 120, and thigh crease 125. For use with the male patient, UCH 200 may be positioned and secured to the thigh 105 at the level of the external urethral meatus 110. Similar to use with a female patient, the catheter holding arm 210 may be bent into a position abutting external urethral meatus 110. The semi-rigid portion 215 (FIG. 2) and 220 (FIG. 2) of arm 210 may be positioned perpendicular to the long axis of the patient's urethra.

Catheter holding arm 210 may be positioned in front of external urethral meatus 110 such that the portion of catheter 300 immediately outside the urethra passes between semi-rigid portion 215 and closure portion 220, or the leaves of catheter arm 210. Catheter 300 may be held with adequate friction to allow urodynamic testing to be performed.

If and when voiding studies are to be performed, catheter holding arm 210 can be separated from base portion 205 as the patient voids, and catheter holding arm 210 may be reattached afterward if necessary.

During urethral pressure profile testing, urethral catheter 300 may be withdrawn and advanced by an operator without removing catheter 300 from between the leaves of catheter holding arm 210

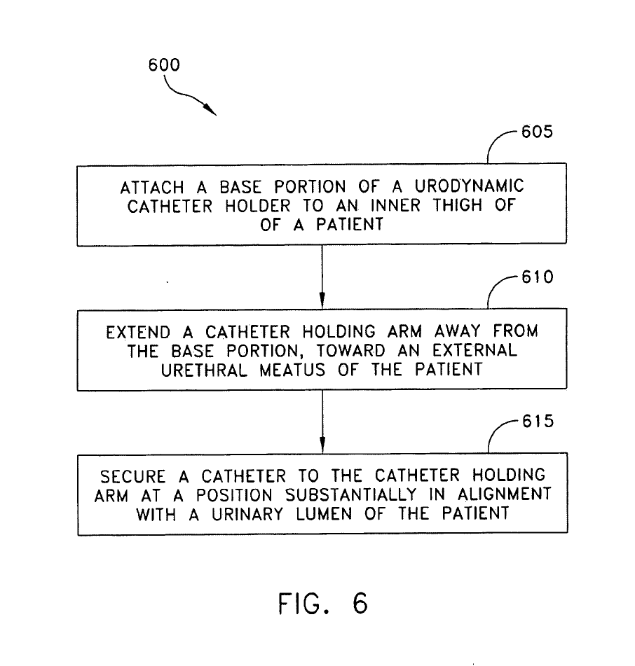

Referring now to FIG. 6, in an exemplary embodiment, a method 600 of holding a urethral catheter may include attaching 605 a base portion of a urodynamic catheter holder to an inner thigh of a patient. Method 600 may further include extending 610 a catheter holding arm away from the base portion, toward an external urethral meatus of the patient. Method 600 may also include securing 615 a catheter to the catheter holding arm at a position substantially in alignment with a urinary lumen of the patient. In an embodiment, method 600 may be used when performing a urodynamic testing procedure.

In addition, the catheter holding arm may be folded back on top of itself after securing the catheter. In other words, with a first end of the catheter holding arm extending from the base portion, a second end of the catheter holding arm may be selectively positionable with respect to the base portion. This positioning may include, but is not limited to, after attaching a first side of the base portion against an inner thigh of the patient, extending the catheter holding arm from a first configuration adjacent to a second side of the base portion to an angle of about 90° with respect to the second side of the base portion. Using a closure portion, the urethral catheter may be attached to the catheter holding arm. The closure may be disposed away from the first end of the catheter holding arm. The catheter holding arm attaches to the urethral catheter in front of the urethra during the urodynamic testing procedure. In an embodiment, this attachment of the urethral catheter to the catheter holding arm is directly in front of, and adjacent to, the exit point of the catheter from the external urethral meatus. Furthermore, and in one embodiment, folding the second end toward the first end of the catheter holding arm may effectively tighten the grip on the catheter and prevent the catheter holding arm from poking into the patients opposite thigh.

What is claimed is:

1. A catheter holding device for use with a urethral catheter in a urodynamic testing procedure, the device comprising a catheter holding arm for holding the urethral catheter in front of the urethra during the urodynamic testing procedure.

2. The device in accordance with claim 1, wherein the malleable arm includes at least one portion of hook-and-loop material configured to allow the urethral catheter to pass between matingly secured layers of hook-and-loop material used to construct the malleable arm.

3. The device in accordance with claim 1, such that the catheter holding arm is removably attached to a base portion.

4. The device in accordance with claim 3, wherein the catheter holding arm is removably attached to the base element using matingly compatible surfaces of hook-and-loop material disposed on the catheter holding arm and the base portion.

5. The device in accordance with claim 1, further comprising a base portion configured for attachment to a patient's inner thigh.

6. The device in accordance with claim 5, such that the base portion includes at least one of an adhesive material and a circumferential strap for attachment to the patient's thigh.

7. The device in accordance with claim 1, wherein the catheter holding arm is configured to allow movement of the urethral catheter with reasonable force with respect to the urethra and bladder without removing the urethral catheter from the catheter holding arm.

8. The device in accordance with claim 1, wherein the catheter holding arm is configured for removal and reinsertion of the urethral catheter therefrom during the urodynamic testing procedure.

9. The device in accordance with claim 1, further comprising a securing portion for attaching a second catheter.

10. The device in accordance with claim 9, wherein the securing portion is positioned against a thigh of a patient.

11. The device in accordance with claim 9, wherein a matingly compatible surface of hook-and-loop material of the base portion is configured to fold over a second catheter for securing the second catheter to the base portion.

12. The device in accordance with claim 11, wherein an exposed surface of hook-and-loop material over the second catheter is matingly compatible with the hook-and-loop surface of the base portion.

13. The device in accordance with claim 1, wherein the catheter holding arm is configured for use with a male patient.

14. The device in accordance with claim 1, wherein the catheter holding arm is configured for use with a female patient.

15. The device in accordance with claim 1, wherein the catheter holding arm includes a malleable material.

16. The device in accordance with claim 1, wherein the catheter holding arm has a first end and a second end, the first end extends from a base portion in attachment to a patient, and a malleable material included between the first end and the second end allows folding of the catheter holding arm between the first end and the second end.

17. A method of holding a urethral catheter, the method comprising:
attaching a base portion of a urodynamic catheter holder to an inner thigh of a patient;
extending a catheter holding arm away from the base portion, toward an external urethral meatus of the patient; and
securing a catheter to the catheter holding arm at a position substantially in alignment with a urinary lumen of the patient.

18. A method in accordance with claim 17, further comprising folding the catheter holding arm between a first end extending from the base portion and a second end disposed away from the first end.

19. A catheter holding device for use with a urethral catheter in a urodynamic testing procedure, the device comprising:
a base portion having a first side and a second side, the first side configured for attachment to an inner thigh of a patient; and
a catheter holding arm having a first end and a second end, the first end extending from the second side of the base portion, the second end selectively positionable with respect to the base portion, and a closure portion for attaching the urethral catheter to the catheter holding arm, the closure disposed away from the first end, whereby the catheter holding arm attaches to the urethral catheter in front of the urethra during the urodynamic testing procedure.

20. A catheter holding device in accordance with claim 19, further comprising a malleable material included between the first end and the second end, whereby the malleable material allows folding of the catheter holding arm between the first end and the second end.
